(19) United States
(12) Patent Application Publication
(10) Pub. No.: US 2025/0288340 A1
(43) Pub. Date: Sep. 18, 2025

(54) BONE CEMENT MIXING SYSTEM HAVING PUNCTURE CONNECTION AND MULTIFUNCTIONAL PLUNGER

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/075,499

(22) Filed: Mar. 10, 2025

(30) Foreign Application Priority Data
Mar. 15, 2024 (EP) 24163705.7

(51) Int. Cl. A61B 17/88

(52) U.S. Cl. CPC A61B 17/8833; A61B 17/8822; A61B 2017/8838

(57) ABSTRACT

A device for producing bone cement, comprising: a base part, which comprises a container having a carrier for accommodating a vessel containing monomer liquid, a bottom part having a collection region, a hollow pin having a tip, and a plunger having a feedthrough for accommodating the hollow pin; and a cartridge for accommodating a PMMA bone cement powder, wherein the cartridge comprises a septum which delimits the cartridge to the outside; wherein the plunger is configured to accommodate the tip of the hollow pin in the feedthrough when the device is in a basic configuration and to release the tip of the hollow pin from the feedthrough and simultaneously release a monomer liquid from a vessel when the cartridge is pressed onto the plunger.

### CROSS-REFERENCE TO RELATED APPLICATIONS

This application claims priority pursuant to 35 U.S.C. 119(a) to European Application No. 24163705.7, filed Mar. 15, 2024, which application is incorporated herein by reference in its entirety.

### FIELD OF THE INVENTION

The present invention relates to the field of medical technology, in particular devices for producing a PMMA bone cement and for storing components thereof.

### BACKGROUND OF THE INVENTION

Polymethylmethacrylate bone cements are based on the fundamental work of CHARNLEY. Polymethylmethacrylate bone cements are usually composed of a liquid monomer component and a powder component. The monomer component can contain, for example, the monomer methyl methacrylate and an activator (e.g., N,N-dimethyl-p-toluidine) dissolved therein. The powder component, also referred to as bone cement powder, can comprise one or more polymers which can be produced on the basis of methyl methacrylate and comonomers, such as styrene, methyl acrylate or similar monomers, by polymerization, preferably suspension polymerization. The powder component can further comprise a radiopaque material and an initiator, such as dibenzoyl peroxide. When the powder component is mixed with the monomer component, a plastically deformable dough, the actual bone cement dough, can be produced as a result of swelling of the polymers of the powder component in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough increases until the cement dough solidifies.

Polymethylmethacrylate bone cements can be mixed in suitable mixing beakers with the aid of spatulas by mixing the powder component with the monomer liquid. Air bubbles may be incorporated in the bone cement dough, which can negatively influence the mechanical properties of the hardened bone cement.

To avoid air inclusions in the bone cement dough, a plurality of vacuum cementing systems have been described, of which the following are mentioned by way of example: U.S. Pat. Nos. 6,033,105 A, 5,624,184 A, 4,671,263 A, 4,973,168 A, 5,100,241 A, WO 99/67015 A1, EP 1 020 167 A2, U.S. Pat. No. 5,586,821 A, EP 1 016 452 A2, DE 36 40 279 A1, WO 94/26403 A1, EP 1 005 901 A2, U.S. Pat. No. 5,344,232 A.

A further development in cementing technology are cementing systems in which both the cement powder and the monomer liquid are already packaged in separate compartments of the mixing systems and are mixed with one another in the cementing system only immediately before application of the cement. Such closed full-prepacked mixing systems are described in the patents documents EP0380867B1, EP0796653B1, EP0692229B1, U.S. Pat. Nos. 5,997,544A, 6,709,149B1, DE69812726T2 and U.S. Pat. No. 5,588,745A, WO9416951A1, DE19718648A1, EP1741413B1, EP3054880B1, DE102009031178B3, U.S. Pat. No. 8,662,736B2, EP2269718B1, EP2281532B1 and EP3093067B1.

There are some challenges with full-prepacked mixing systems. It is desirable to ensure reliable, reproducible transfer of the monomer liquid into the polymethylmethacrylate bone cement powder and to avoid uncontrolled mixing of the components during storage. It is advantageous if, during transport and storage of the mixing system, the cement powder cannot penetrate into the conduit means for transferring the monomer liquid. Otherwise, the conduit means can become blocked during monomer transfer due to swelling and sticking. Blockage of the conduit means by swollen, sticking bone cement could interrupt the transfer of the monomer liquid and prevent the mixing of the cement components in the predefined mixing ratio.

In the full-prepacked mixing systems according to EP0796653B1, U.S. Pat. No. 6,709,149B1, EP1741413B1, EP3054880B1, EP2269718B1 and EP2281532B1, the monomer transfer is carried out by applying an external vacuum to the cartridge via a vacuum port and a porous plate. This means that the monomer liquid is sucked into the cartridge by the applied vacuum. In the mixing system described in EP3093067B1, either the monomer liquid can be transferred into the cartridge by means of a vacuum, or alternatively the monomer liquid can be transferred by pressure by means of a manually operated pump plunger integrated into the mixing system.

According to EP2281532B1, the blockage of the conduit means for transferring the monomer liquid is prevented by using a mesh or pore disc permeable to the monomer liquid to block the cement powder particles, the mesh or pore disc being part of a special nozzle that generates a sharp monomer liquid jet during transfer, which jet displaces swollen cement particles so that a complete transfer of the monomer liquid into the bone cement powder is possible.

EP1741413B1 discloses a method for bringing a powder and a liquid component into contact, wherein a liquid container is opened by the penetration of a cannula into a wall portion of the liquid container, which wall portion closes a lateral hole in an inner tube element in the liquid container.

Patent document EP3636338B1 discloses a mixing system comprising a connecting cylinder configured to be selectively coupled to and removable from a mixing chamber, and wherein a valve is configured to seal the connection between the connecting cylinder and the valve when they are selectively coupled to one another and is configured to seal the mixing chamber when the connecting cylinder is removed from the mixing chamber. EP3490700B1 discloses a similar mixing system.

### SUMMARY OF THE INVENTION

An object of the present invention is to solve one or more of the problems described above and further problems of the prior art. For example, the invention provides a device for storing and mixing bone cement, said device allowing easy and reliable mixing of a monomer liquid with a powder component of a PMMA bone cement in order to produce a ready-to-use PMMA bone cement dough therefrom. The devices according to the invention can comprise several parts which can be easily connected to one another and disconnected from one another again, while allowing reliable and secure transfer of a monomer liquid from one part of the device into the other part of the device. Leakage of monomer liquid or ingress of contaminants can be prevented by a self-sealing connection system. The device according to the invention can offer easy handling. A monomer liquid and a powder component of a PMMA bone cement can be stored separately in the device, brought together in a controlled manner and mixed together. The device can release a monomer liquid from a closed vessel in an interior of the device, in particular by means of a movable plunger, which can simultaneously also serve to establish a fluidic connection between one part of the device and another part of the device. The device can perform these functions while securely standing on a supporting surface. The monomer liquid can be completely transferred, wherein clogging of the device by clumped powder can be prevented.

These objects are achieved by the methods, devices, kits and medical uses described herein, in particular those which are described in the claims. Preferred embodiments of the invention are described below.

A first embodiment relates to a device for producing bone cement, comprising a base part,

which comprises a container having a carrier for accommodating a vessel containing monomer liquid, a bottom part having a collection region, a hollow pin having a tip, and a plunger having a feedthrough for accommodating the hollow pin, and a cartridge for accommodating a PMMA bone cement powder, wherein the cartridge comprises a septum which delimits the cartridge to the outside; wherein the plunger is configured to accommodate the tip of the hollow pin in the feedthrough when the device is in a basic configuration and to release the tip of the hollow pin from the feedthrough and simultaneously release a monomer liquid from a vessel when the cartridge is pressed onto the plunger.

A second embodiment relates to a device according to the first embodiment, wherein the device is designed and configured to form a fluidic connection between the base part and the cartridge by piercing the septum by means of the hollow pin, in order to transfer a monomer liquid from the collection region into the cartridge.

A third embodiment relates to a device according to the first or second embodiment, wherein the vessel is a glass ampule or a film bag.

A fourth embodiment relates to a device according to the second or third embodiment, wherein the carrier is designed and configured to bring a head of a glass ampule into contact with the plunger.

A fifth embodiment relates to a device according to one of the preceding embodiments, wherein the base part comprises a spring which is arranged and configured to movably support the plunger within the base part.

A sixth embodiment relates to a device according to one of the preceding embodiments, wherein the base part comprises a stop in order to limit the movement of the plunger and/or the cartridge in the base part.

A seventh embodiment relates to a device according to one of the preceding embodiments, wherein the device comprises a filter, wherein the filter is arranged and designed to prevent the ingress of particles from the base part into the cartridge.

An eighth embodiment relates to a device according to the seventh embodiment, wherein the cartridge comprises a port for connection to a vacuum source in order to allow a monomer liquid to be transferred from the collection region into the cartridge by means of a pressure difference.

A ninth embodiment relates to a device according to one of the preceding embodiments, wherein the base part comprises a ventilation opening, wherein the ventilation opening is preferably designed as a valve.

A tenth embodiment relates to a device according to one of the preceding embodiments, wherein the septum comprises a rubber-elastic material.

An eleventh embodiment relates to a device according to one of the preceding embodiments, wherein the base part is configured to allow a monomer liquid to be transferred from the container into the collection region by free gravitational flow.

A twelfth embodiment relates to a device according to one of the preceding embodiments, wherein the container is arranged at an angle to the bottom part which is 20° to 80°, preferably 21° to 55°.

A thirteenth embodiment relates to a device according to one of the preceding embodiments, wherein the cartridge can be detachably connected to the base part.

A fourteenth embodiment relates to a device according to one of the preceding embodiments, wherein the conduit element is configured to establish a fluidic connection between the collection region and the hollow pin.

A fifteenth embodiment relates to a device according to one of the preceding embodiments, wherein the hollow pin is mounted in a cylindrical holder, wherein the holder is arranged in the collection region.

A sixteenth embodiment relates to a device according to one of the preceding embodiments, wherein the collection region comprises a recess, and wherein the hollow pin is preferably fluidically connected to the lowest point of the recess.

A seventeenth embodiment relates to a device according to one of the preceding embodiments, wherein the device is designed and configured to transfer a monomer liquid from the base part into the cartridge while the base part stands on a supporting surface such that the bottom part is oriented in the direction of the supporting surface.

### BRIEF DESCRIPTION OF THE DRAWINGS

FIG. 1 shows the cross section of a base part of a device according to the invention;

FIG. 2 shows the cross section of a cartridge of a device according to the invention;

FIG. 3 shows the cross section of a base part in which two glass ampules are accommodated in a carrier;

FIG. 4 shows the cross section of a base part according to FIG. 3, in which base part the two glass ampules are broken open by movement of a plunger toward the bottom of the base part in order to discharge monomer liquid from the glass ampules into a collection region;

FIG. 5 shows a device according to the invention, wherein a base part is in contact with a cartridge, wherein, as a result of pressing the cartridge against a plunger of the base part, a hollow pin of the base part pierces a septum of the cartridge;

FIG. 6 shows a device according to the invention, wherein monomer liquid is discharged from a collection region of the base part into the cartridge via a hollow pin by means of a pressure difference between the base part and the cartridge;

FIG. 7 shows a cartridge during discharging of a bone cement dough; and,

FIG. 8 shows an isometric view of a device according to the invention, wherein the base part can be connected to the cartridge by means of a contact region of the base part.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products, devices, kits and uses described herein, and vice versa. All such considered combinations are not explicitly listed in all instances, simply in order to keep the description brief. Technical solutions known to be equivalent to the features described herein are also intended in principle to be encompassed by the scope of the invention.

A first embodiment relates to a device for producing bone cement, comprising a base part, which comprises a container having a carrier for accommodating a vessel containing monomer liquid, a bottom part having a collection region, a hollow pin having a tip, and a plunger having a feedthrough for accommodating the hollow pin, and a cartridge for accommodating a PMMA bone cement powder, wherein the cartridge comprises a septum which delimits the cartridge to the outside, wherein the plunger is configured to accommodate the tip of the hollow pin in the feedthrough when the device is in a basic configuration and to release the tip of the hollow pin from the feedthrough and simultaneously release a monomer liquid from a vessel when the cartridge is pressed onto the plunger.

The device according to the invention can comprise two parts that can be connected to one another, namely a base part and a cartridge. The base part comprises a container for accommodating a monomer liquid. The container comprises a carrier for accommodating a vessel containing monomer liquid, for example a glass ampule or a bag. Such glass ampules or bags are used as commercially available packagings for monomer liquid of a PMMA bone cement. The container can be prefilled with monomer liquid, for example packaged gas-tight in a glass ampule or in a bag. Alternatively, the container can be provided empty and be fillable with monomer liquid by the user, for example by inserting a glass ampule containing monomer liquid into the container.

The cartridge can also be provided empty or prefilled with PMMA bone cement powder. The base part can comprise a transparent region. For example, the container can comprise a transparent region. The collection region can likewise comprise a transparent region. This can allow a user of the device to visually verify, from the outside, the complete drainage of monomer liquid from the container into the collection region. Furthermore, the base part can comprise a fill level marking. Such a fill level marking can, for example, be arranged at the position of a desired liquid level of the collection region, which desired liquid level can be achieved by a complete transfer of a monomer liquid from the container into the collection region.

The base part can be designed to stand on a supporting surface. The base part comprises a bottom part, which can have a flat exterior. The bottom part comprises a collection region. The collection region is preferably designed and configured to accommodate monomer liquid. For example, the base part can be configured to release monomer liquid from a vessel, such as a glass ampule or a film bag, and to transfer it into the collection region by gravitational flow. The collection region can comprise a recess, for example a concave curvature, comparable to a watch glass. The cross section of the recess can be U-shaped or V-shaped. The recess can have a funnel-shaped form.

The collection region preferably has an accommodation capacity for monomer liquid which exceeds the volume of the monomer liquid contained or accommodable in the container. For example, the collection region can have a volume which is at least 1.1 times the volume of the monomer liquid contained or accommodable in the container. If, for example, the container comprises a carrier for accommodating commercially available glass ampules containing 10 ml of monomer liquid, the collection region preferably has a volume of at least 11 ml. This applies correspondingly to embodiments which comprise a carrier for accommodating glass ampules containing 5 ml, 20 ml or 30 ml of monomer liquid.

The base part also comprises a hollow pin having a tip. The hollow pin preferably comprises an interior that allows a liquid to flow through the hollow pin.

The interior can have a diameter of 1.0 mm to 3.0 mm. The hollow pin can comprise outlet openings having a diameter of less than 1.5 mm and particularly preferably less than 1.0 mm. As a result of a small diameter of the hollow pin, a sharp jet can be formed when a monomer liquid is transferred from the collection region into the cartridge, which jet can optionally displace smaller particles of bone cement powder. In this way, clogging of the hollow pin can be prevented. The hollow pin can comprise a beveled tip, similarly to a ground cannula, with an outlet opening of the hollow pin preferably being located in a bevel of the tip. The hollow pin can comprise a round tip. One or more openings connected to the interior of the hollow pin can be arranged near the tip. Furthermore, the interior of the cavity can comprise a shoulder on which a porous peg permeable to liquids is supported. This peg can serve as a filter, as described below. The interior of the hollow pin can be tapered toward the shoulder. The shoulder can be configured to hold the peg in the hollow pin. The hollow pin can be made of plastic or metal, e.g. stainless steel. Examples of usable plastics include polyamide 12, polyamide 6, polyamide 66 and polyethersulfone. The hollow pin, particularly the tip of the hollow pin, can be covered by a removable protective cap.

The cartridge comprises a septum which delimits the cartridge to the outside. The septum is preferably arranged in a region of the cartridge which is designed and configured for connection to the base part. The septum can be pierced by means of the hollow pin of the base part. In a configuration of the device in which the septum is pierced by the hollow pin, a fluidic connection can form between the base part and the cartridge. This makes it possible to transfer a monomer liquid from the collection region of the base part into the cartridge. At the same time, the self-sealing function of the septum can prevent monomer liquid from escaping from the device. This also applies if the hollow pin is pulled out of the septum again, because the puncture site can be sealed automatically due to the restoring force of the septum. Furthermore, this can prevent contaminants from entering the device from the outside. In one embodiment, the device is designed and configured to form a fluidic connection between the base part and the cartridge by piercing of the septum by means of the hollow pin, in order to transfer a monomer liquid from the collection region into the cartridge.

In some embodiments, the septum comprises a flexible, self-sealing polymer. The polymer can comprise a polyhalogenated olefin, a silicone, rubber, butyl rubber, ethylene propylene diene rubber (EPDM), or a similar elastomer. The septum is preferably configured to seal the cartridge in a liquid-tight manner after the septum has been pierced by means of the hollow pin. The septum is preferably stretchable and exhibits rubber-elastic properties, as is known, for example, from septa in chemical bottles. This allows the septum to heal itself after a puncture, i.e. to self-seal.

The septum preferably comprises a biocompatible material. The septum can comprise a circular shape. In one embodiment, the septum comprises a material which can be pierced with a medical injection cannula according to ISO 7864:2016 with a force of less than 100 N. The septum is preferably connected to the cartridge in a liquid-tight manner. In one embodiment, the septum is designed and configured to seal a puncture site in the septum in a liquid-tight manner after a puncture and subsequent removal of the hollow pin. This can be achieved by the restoring force of a rubber-elastic material of the septum.

The cartridge may comprise a guide element which facilitates insertion of the hollow pin into a desired position of the septum, e.g., an insertion slope.

The septum can be mounted in a holder. The holder can be detachably connected to the cartridge. The holder of the septum can be designed as a plunger. This plunger can be designed and configured to discharge bone cement dough from the cartridge. The holder for the septum can be arranged in a cartridge head. The cartridge head can be designed as a releasable closure of the cartridge.

The cartridge and the base part preferably can be stored separately from one another, i.e. they are designed as separate sub-elements that can be detachably connected to one another. This can prevent fatigue of the restoring force of the septum during storage and facilitate the self-closing of the septum. An unwanted leakage of a monomer liquid from the cartridge after the removal of the hollow pin from the septum can be prevented. Furthermore, the separate storage has the advantage that packaging materials with small dimensions can be used.

The device also comprises a plunger. The plunger can be arranged within the base part. The plunger can have a feedthrough for accommodating the hollow pin. The feedthrough can extend from an interior of the base part to the outside. The plunger can be designed such that, when the device is in a basic configuration, the plunger accommodates the tip of the hollow pin in the feedthrough. In such a basic configuration of the device, no external force is exerted on the device. In this state, the tip of the hollow pin is arranged inside the plunger so that a user is protected from injury by contact with the tip. In one embodiment, the plunger is configured to release the tip of the hollow pin from the feedthrough when the cartridge is pressed onto the plunger. This makes it possible to apply pressure to the plunger by means of the cartridge in order to release the hollow pin and pierce the septum of the cartridge. In the process, the tip of the hollow pin can protrude from the feedthrough. This allows the cartridge and the base part to be fluidically connected to each other in an injury-proof way in order for a monomer liquid to be received into the cartridge.

The plunger is preferably configured to be pushed into the base part by the cartridge when the cartridge is connected to the base part. By the exertion of pressure against the plunger by the cartridge, the plunger can accordingly be moved.

Furthermore, the plunger is configured to release a monomer liquid from a vessel when the cartridge is pressed onto the plunger. For this purpose, the plunger can be arranged movably in the base part. As a result of pressure on the plunger, the plunger can be moved preferably toward the collection region of the base part. The plunger can therefore be moved toward the interior of the base part or toward a standing surface of the base part. As a result of such a movement of the plunger, the plunger can be brought into contact with a vessel containing monomer liquid in order to open said vessel. For example, the plunger may be able to be moved against the head of a glass ampule accommodated in the carrier of the container. This allows the ampule head to be broken off in order to release a monomer liquid from the glass ampule. Similarly, the plunger can be configured to open a bag containing monomer liquid when the plunger is moved into the interior of the base part by pressure from the cartridge. The plunger can comprise an opening means, such as a pin or a knife, in order to open a film bag containing monomer liquid. For example, the plunger can comprise a peripherally extending comb structure by means of which a film bag can be opened. The opening means can be designed as a sharp outer edge of the plunger. The opening means can be made of plastic or metal.

According to the invention, the plunger is configured to accommodate the tip of the hollow pin in the feedthrough when the device is in a basic configuration and to release the tip of the hollow pin from the feedthrough and simultaneously release a monomer liquid from a vessel when the cartridge is pressed onto the plunger.

In one embodiment, the carrier is designed and configured to bring a vessel containing a monomer liquid into contact with the plunger. In one embodiment, the carrier is designed and configured, for example, to bring a head of a glass ampule into contact with the plunger. For example, the carrier may make it possible to arrange a glass ampule within the travel path of the plunger. This means that the movement of the plunger can bring the plunger into contact with the glass ampule in order to exert pressure on the glass ampule and thereby break the glass ampule open. The plunger can therefore cause a release of monomer liquid from a glass ampule. Correspondingly, in some embodiments the carrier is designed and configured to bring a film bag into contact with the plunger. Thus, a film bag can be opened as a result of movement of the plunger, in order to discharge monomer liquid from the film bag. After being discharged from a container, such as a glass ampule or a film bag, the monomer liquid can be accommodated in the collection region.

In one embodiment, the base part comprises a spring which is arranged and configured to movably support the plunger within the base part. This allows the plunger to be held in a position in which the tip of the hollow pin is accommodated in the feedthrough of the plunger, as described above, and is thus held within the plunger in a way that prevents injury. The spring can be a coil spring. In one embodiment, the spring surrounds the hollow pin along a portion of the hollow pin located outside the feedthrough of the plunger. The spring can be arranged between the bottom part and the plunger.

In an embodiment in which the hollow pin is mounted in a cylindrical holder, as described herein, the spring can be arranged between this holder and the plunger. The spring can facilitate the separating of the base part and the cartridge. Furthermore, the tip of the hollow pin can be automatically moved back into the cavity by the spring as soon as external force no longer acts on the plunger, e.g., when the user no longer actively presses the cartridge against the base part.

In one embodiment, the base part comprises a stop. The stop can be designed and configured to limit the movement of the plunger and/or the cartridge in the base part. The stop can be configured to hold the plunger in the base part. In an embodiment in which the base part comprises a spring, the stop can limit the travel path of the plunger in the direction of the force applied by the spring. This can prevent the plunger from being pushed out of the base part by the spring. In one embodiment, the stop is configured such that, when the device is in a basic configuration, the stop holds the plunger in a position in which the plunger is flush with an outer surface of the base part. In one embodiment, the stop is configured to hold the plunger in a position in which the tip of the hollow pin is accommodated in the feedthrough of the plunger. Furthermore, the stop can be designed and configured to limit the travel path of the cartridge into the interior of the base part. This allows the cartridge to be placed in a desired position on the base part without the cartridge and base part moving relative to each other. A releasable connecting element can be provided in order to hold the cartridge on the base part in a desired position. In such a desired position, the cartridge can be flush with the stop. This connecting element can prevent the cartridge from being pushed out of the base part by the spring. Thus, in such an embodiment, the user does not have to compensate for the pressure of the spring with their own force, during use of the device, in order to hold the cartridge in position. The cartridge can have a projection designed and configured to engage with stop.

In one embodiment, the device comprises a filter. The filter can be arranged in the base part. For example, the filter can be arranged between the container and the hollow pin. The filter is preferably designed and configured to prevent an ingress of particles from the base part into the cartridge. For example, the filter can be arranged and configured to retain glass fragments from a glass ampule broken open in the base part. The filter can be, for example, a metal wire mesh or a mesh made of a plastic material. The filter can also be a metal or plastic disc provided with expediently dimensioned holes. Furthermore, open-pore foams, such as Porex® foam, can be used. Preferably, the filter should be made of a material which is not attacked by the monomer liquid contained in an accommodable glass ampule.

In one embodiment, the cartridge comprises a port for connection to a vacuum source. Such a port can enable the transfer of a monomer liquid from the collection region into the cartridge by means of a pressure difference. For example, by means of a vacuum pump that can be connected to the cartridge, a negative pressure can be created in the cartridge in order to receive monomer liquid from the base part into the cartridge via the fluidic connection which can be established by piercing of the septum by means of the hollow pin. The port is preferably spaced from the septum such that no monomer liquid can enter the port, particularly in a configuration in which the base part is standing on a supporting surface. For example, the septum and the port can be arranged on opposite sides of the cartridge.

In one embodiment, the container comprises an ampule breaker. The ampule breaker can be configured to open a glass ampule within the container. The container can also comprise another means for releasing a monomer liquid from a vessel, e.g. from a film bag. This can be, for example, a pin suitable for piercing a film bag, as described in more detail elsewhere herein.

In one embodiment, the ampule breaker is designed and configured to open a glass ampule within the container by bending the container relative to the bottom part. For this purpose, the container can have a flexible housing part which enables a force to be exerted on a glass ampule in the container. The ampule breaker can have a rigid housing part or projection against which a glass ampule can be pressed to break the glass ampule open.

In one embodiment, the container is configured to allow the transfer of a monomer liquid from the container into the collection region by free gravitational flow. “Free gravitational flow” means herein that a monomer liquid can pass from the container into the collection region by the action of gravity alone, without the need for, for example, a pressure difference between the container and the collection region. Preferably, the devices described herein allow complete transfer of the monomer liquid from the container into the collection region by free gravitational flow within 20 seconds. The monomer liquid can then be transferred from the collection region into the cartridge by suction, as described herein.

In one embodiment, the container is arranged at an angle to the bottom part which is 20° to 80°, preferably 21° to 55°. This angle is defined by the inside of the container and the inside of the bottom part, where the container and the bottom part merge into one another toward the bottom part (i.e. toward the “underside” of the base part) at a vertical side wall of the bottom part. This corresponds to the point at which the monomer liquid flows from the container into the collection region when the device is used as intended. The container is preferably arranged opposite the bottom part, i.e. oriented “upwards” when the device is used as intended, in order to allow the monomer liquid to drain into the collection region. For example, the container can be arranged in relation to the rest of the base part similarly to a wing of a wing nut. The base part can comprise, for example, two or more containers. In one embodiment, the base part comprises two containers arranged in a Y shape. The plunger can be arranged between the containers, i.e., between the two Y-arms. The container can be arranged so that, when the base part is in a configuration in which the base part stands on an underlying surface with the bottom part oriented in the direction of the underlying surface, the container is located above the collection region. This allows reliable drainage of monomer liquid from the container into the collection region.

In one embodiment, a longitudinal axis of the container is arranged at an angle to an axis that runs perpendicularly to a standing surface of the bottom part, which angle is 20° to 80°, preferably 21° to 55°. At an angle of 21° to 55°, the formation of a meniscus by the monomer liquid can be particularly prevented.

In one embodiment, the cartridge can be detachably connected to the base part. For this purpose, appropriate connecting elements, such as a thread, can be provided on the cartridge and on the base part. The connecting elements can be arranged in such a way that they allow a connection of the hollow pin to the septum. Furthermore, the device can comprise a guide element which facilitates the connection of the base part to the cartridge in a desired orientation. Preferably, the base part and the cartridge can be connected to one another in such a way that the plunger and the septum are flush with one another. For this purpose, for example the base part can comprise a protruding region which is designed and configured to engage in the cartridge.

In one embodiment, the base part comprises a conduit element, wherein the conduit element is configured to establish a fluidic connection between the collection region and the hollow pin. The conduit element enables a transfer of monomer liquid from the collection region into the hollow pin, preferably a transfer by free gravitational flow. The conduit element can be designed, for example, as a hose, tube or feedthrough. In an embodiment in which the hollow pin is mounted in a cylindrical holder, as described herein, the conduit element can be designed as a feedthrough in the holder.

In one embodiment, the device is designed and configured to transfer a monomer liquid from the base part into the cartridge while the base part stands on a supporting surface such that the bottom part is oriented in the direction of the supporting surface. The containers and the cartridge preferably point “upwards”, i.e. in a direction away from the underlying surface, and against the direction of gravity. The monomer liquid can therefore flow from the container into the collection region by the effect of gravity.

In some embodiments, the cartridge comprises a PMMA bone cement powder. The PMMA bone cement powder is preferably arranged in an interior of the cartridge.

In some embodiments, the container comprises a monomer liquid. The monomer liquid is preferably arranged in an interior of the container. The monomer liquid can be arranged in a glass ampule or in a sealed film bag within the container.

In some embodiments, the container also comprises a filter permeable to a monomer liquid. The filter is preferably arranged and configured to prevent an ingress of glass splinters from the container into the cartridge. The device can be configured to accommodate a glass ampule containing monomer liquid. The device can also be configured to break open a glass ampule containing monomer liquid. The glass ampule can be accommodated and broken open in particular within the container. For this purpose, the container can comprise a receptacle for a glass ampule. The container can comprise a means for breaking open a glass ampule. For example, the container can comprise a projection arranged and configured to break off the head of a glass ampule.

The filter can be configured to retain fragments of a glass ampule in the container. This can prevent glass splinters from getting into the cartridge. This can prevent contamination of bone cement dough with glass splinters.

In some embodiments, the device comprises a mixing rod. The mixing rod is preferably configured for mixing monomer liquid and PMMA bone cement powder. The mixing rod can comprise blades. The blades can facilitate a homogeneous mixture of monomer liquid and PMMA bone cement powder. The blades are preferably arranged at an end of the mixing rod which is arranged within the cartridge. The mixing rod can comprise one, two or more than two blades. The mixing rod can comprise a handle at an end of the mixing rod which is preferably arranged outside the cartridge. The mixing rod is preferably movably arranged in the cartridge.

The mixing rod can comprise a discharge tube. The discharge tube can be designed and configured to discharge a bone cement dough mixed in the device (i.e. a bone cement dough mixed from monomer liquid and PMMA bone cement powder). Preferably, the discharge tube is closable. This can prevent bone cement dough from accidentally escaping from the cartridge during mixing. The discharge tube can be designed as a cavity within the mixing rod. In some embodiments, the discharge tube can be closed by a removable core. Accordingly, the mixing rod can have a detachable, rod-shaped core, for example a metal core. The core can also improve the strength of the mixing rod.

The mixing rod can have a predetermined breaking point for breaking off the mixing rod after the mixing process. The predetermined breaking point is preferably located outside the cartridge so that the mixing rod can be broken off from the outside without opening the cartridge.

In some embodiments, the container also comprises a carrier for accommodating an ampule. Such ampules can be, in particular, glass ampules in which monomer liquid is commercially available. The carrier is preferably arranged and configured to allow an ampule to be broken open and monomer liquid to drain from the ampule through the outlet and the introduction tube by free gravitational flow.

In some embodiments, the container also comprises a means for opening a film bag or a glass ampule within the device. An example of a means for opening a film bag is a pin which is suitable for piercing a film bag. Such a pin can be made of a rigid plastic or of metal, for example. An example of a means for opening a glass ampule is a projection against which the head of a glass ampule can be pressed to break the glass ampule open. A means for opening a glass ampule can comprise a flexible housing part so that the glass ampule can be broken from the outside by hand by deforming the flexible housing part. A means for opening a glass ampule can also comprise a movably arranged element, such as a plunger, which can press the glass ampule against a hard housing part, e.g. a projection, and thereby break the glass ampule open. Means for opening a film bag or a glass ampule are also disclosed in DE19532015A1, WO9718031A1, EP2404864B1 and WO2010012114A1.

In one embodiment, the container comprises a projection arranged in the container, which projection is designed and configured to break open a glass ampule accommodable in the container, and also comprises a carrier for receiving a glass ampule, the carrier being movably arranged in the container and being designed and configured to break open a glass ampule accommodable in the holder by pressing the glass ampule against the projection. The carrier can be designed, for example, as a movable plunger.

In one embodiment, the container comprises a flexible region and a rigid region, the flexible region being configured to move a glass ampule accommodated in the container against the rigid region to break open the glass ampule.

Furthermore, the base part can comprise a ventilation opening. The ventilation opening can be arranged in the container. The ventilation opening can preferably be arranged above the collection region in the base part. Preferably, the ventilation opening is arranged in the base part in such a way that contact with monomer liquid in the collection region is avoided. The ventilation opening can be designed either as a simple opening or as a valve. The opening can be covered on the inside by a gas-permeable porous material so that the ingress of particles from outside into the container can be prevented and at the same time an exchange of air through the porous material is possible. The valve can, for example, be designed to allow air to pass only toward the interior of the container. This can improve the transfer of the monomer liquid into the cartridge, in particular when the transfer is carried out by means of a pressure difference. Furthermore, a unidirectional valve can prevent unwanted leakage of monomer liquid from the device.

A further aspect relates to a method for producing a bone cement dough, wherein the method comprises the following steps: providing a device according to one of the embodiments described herein optionally introducing a vessel containing a monomer liquid into the container; optionally introducing a PMMA bone cement powder into the cartridge; bringing the cartridge into contact with the base part; pressing the cartridge against the plunger so that the tip of the hollow pin is released from the feedthrough of the plunger and the vessel is opened by the plunger; piercing the septum by means of the hollow pin so that a fluidic connection is formed between the collection region and the cartridge; optionally arranging the device such that the monomer liquid is transferred from the container into the collection region by free gravitational flow; transferring the monomer liquid from the container into the cartridge; mixing the monomer liquid with the PMMA bone cement powder in the cartridge and thereby obtaining a bone cement dough.

A further aspect relates to a method for producing a bone cement dough, wherein the method comprises the following steps: providing a device which comprises: a base part with a vessel containing monomer liquid, with a movable plunger having a feedthrough, and with a hollow pin; and a cartridge with a septum and with a PMMA bone cement powder; pressing the cartridge against the plunger so that the tip of the hollow pin is released from the feedthrough of the plunger and the vessel is opened by the plunger; piercing the septum by means of the hollow pin so that a fluidic connection is formed between the collection region and the cartridge; transferring the monomer liquid from the container into the cartridge; mixing the monomer liquid with the PMMA bone cement powder in the cartridge and thereby obtaining a bone cement dough.

A further aspect relates to the use of a device described herein or of the method described above to produce bone cement dough, in particular PMMA bone cement dough. Here, a monomer liquid can be mixed with a PMMA bone cement powder within the device to form a bone cement dough. Such a bone cement dough can be discharged from the device and cured to form PMMA bone cement at a target site.

### EXAMPLES

The invention is further illustrated below using examples which are, however, not to be understood as limiting. It will be apparent to a person skilled in the art that other equivalent means may be used similarly in place of the features described here.

### Detailed Description of the Invention

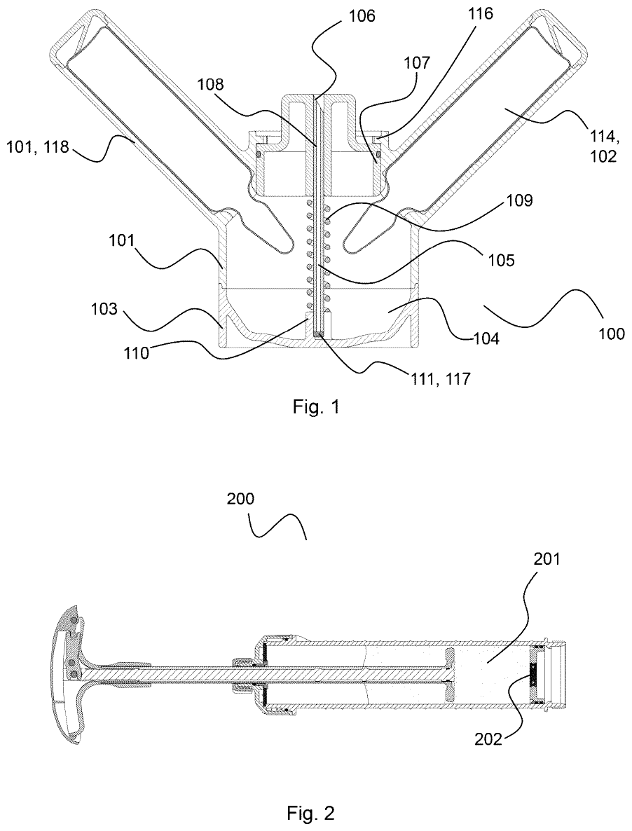

FIG. 1 shows, by way of example, an embodiment of a base part 100 of a device according to the invention. The base part 100 comprises a container 101 having a carrier 118 designed to accommodate a vessel 114 containing a monomer liquid 102. The vessel 114 is designed here as a glass ampule. The container 101 also comprises a bottom part 103 which is designed and configured to stand the base part 100 on a supporting surface. For this purpose, the bottom part 103 can comprise a flat outer surface and/or one or more foot elements. A collection region 104 is located inside the bottom part 103. The collection region 104 is designed to accommodate a monomer liquid which can be discharged from the container 101. Within the base part 100 there is a hollow pin 105 which extends from the collection region 104 to a side opposite from the collection region. In the base part 100 there is also a plunger 107 which is movably mounted on a spring 109. The plunger comprises a feedthrough 108 designed as a cylindrical cavity. The feedthrough is designed and configured to accommodate the hollow pin 105. The hollow pin comprises a tip 106 which is accommodated in the cavity when the base part is in a basic state. The hollow pin 105 is mounted, at an end of the hollow pin which is opposite the tip 106, in a holder 110 located in the bottom part 103. The holder 110 comprises a conduit element 111 which here is in the form of a through-hole through the holder 110. The conduit element 111 establishes a fluidic connection between the collection region 104 and the hollow pin 105. A filter 117 is arranged on the conduit element 111, which filter can prevent the ingress of glass fragments from the collection region 104 into the hollow pin 105. The base part is in a basic configuration here, in which no external force is applied to the plunger 107. Here the spring 109 keeps the plunger 107 in a position in which the tip 106 of the hollow pin 105 stays in the feedthrough 108 so that the tip 106 does not protrude from the feedthrough.

FIG. 2 shows a cartridge 200 of a device according to the invention. The cartridge 200 is designed and configured to accommodate a PMMA bone cement powder 201. The cartridge 200 comprises a septum 202 made of a rubber-elastic material, which delimits the cartridge to the outside. The septum 202 is connected to the housing of the cartridge in a liquid-tight manner. The cartridge is designed and configured to be brought into contact with the base part 100 in order to receive a monomer liquid from the base part 100 into the cartridge.

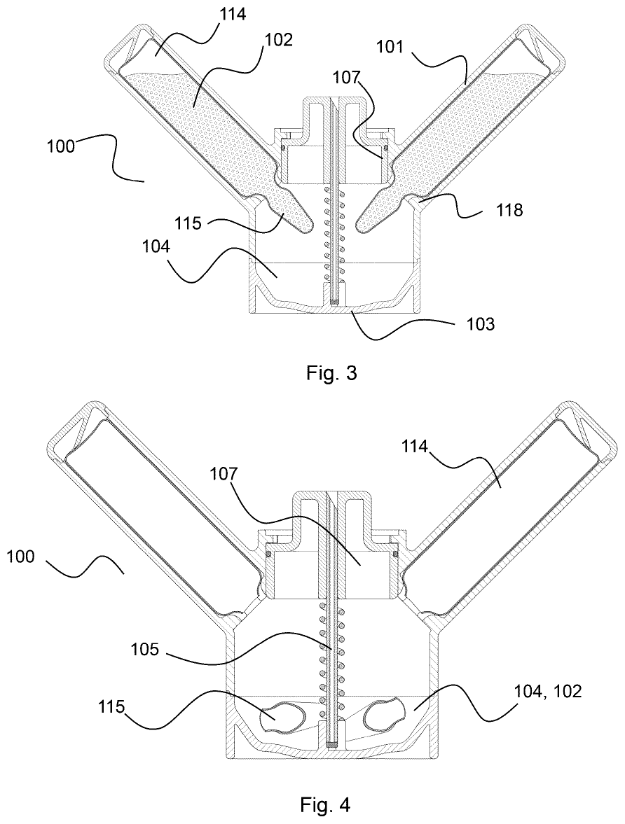

FIG. 3 shows a base part 100 having a container 101, which comprises a carrier 118 for accommodating a glass ampule 114 having an ampule head 115. The carrier 118 holds the glass ampule 114 in a position in which the plunger 107, by its movement toward the bottom part 103, can break off the ampule head 115 and thereby release the monomer liquid 102 from the glass ampule 114. The monomer liquid 102 can drain into the collection region 104 by free gravitational flow.

FIG. 4 shows a base part 100 according to FIG. 3, in which base part the ampule head 115 is broken off by the movement of the plunger 107 in order to release monomer liquid 102 from the glass ampule 114 and to discharge said monomer liquid into the collection region 104. The monomer liquid 102 is located in the collection region 104 in fluidic connection with the hollow pin 105.

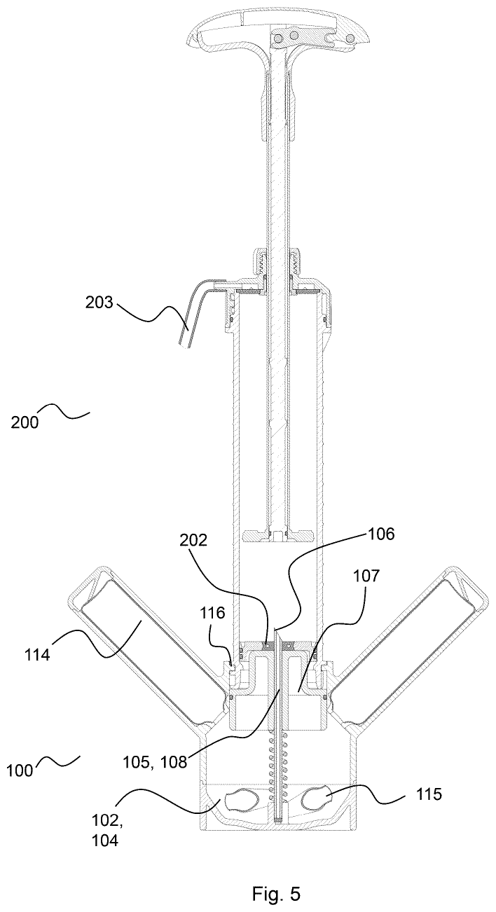

FIG. 5 shows a device according to the invention with a base part 100 which is in contact with a cartridge 200. The base part 100 comprises a stop 116 which is configured to engage with a projection on the cartridge 200. As a result of the cartridge 200 being pressed against the plunger 107, the plunger is moved into the internal space of the base part 100, this movement being limited by the stop 116. The stop 116 can also limit the movement of the plunger 107 outward to prevent the spring 109 from moving the plunger 107 completely out of the base part 100. As a result of the movement of the plunger 107, the ampule head 115 is broken off in order to discharge the monomer liquid 102 into the collection region 104. Furthermore, the movement of the plunger 107 results in the release of the tip 106 of the hollow pin 105 from the feedthrough 108 in order to pierce the septum 202.

The hollow pin 105 is fed through the septum 202 in order to form a fluidic connection between the collection region 104 and the interior of the cartridge 200. Thus, monomer liquid 102 can be transferred from the collection region 104 into the cartridge 200, for example by connecting a vacuum source to the cartridge 200 via a vacuum port 203 provided for such purpose. Thus, the plunger 107 is used both to break open the glass ampule 114 and to establish a fluidic connection of the base part 100 to the cartridge 200.

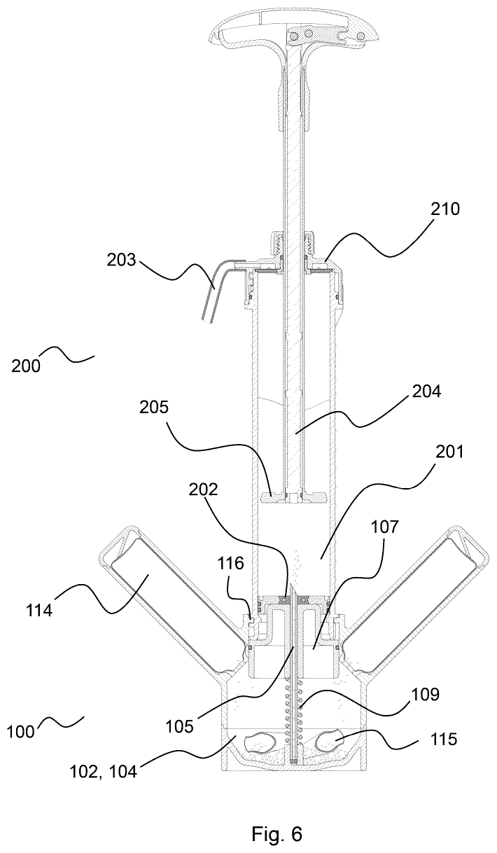

FIG. 6 shows a device according to the invention according to FIG. 5, wherein a glass ampule 114 is broken open by the movement of the plunger 107 pressing against the ampule head 115 and thus has discharged a monomer liquid 102 into the collection region 104. The monomer liquid 102 is subsequently transferred from the collection region 104 into the cartridge 200 through the hollow pin 105 by means of a pressure difference between the base part 100 and the cartridge 200. For this purpose, a vacuum source can be connected to the port 203. The cartridge contains a PMMA bone cement powder 201, which can be mixed with the monomer liquid 102 to form a bone cement dough. For this purpose, the cartridge 200 also comprises a mixing rod 204 with a mixing disc 205. The mixing rod 204 extends through a cartridge head 210 screwed onto the cartridge 200. The port 203 is arranged on the cartridge head 210. The mixing disc 205 is arranged at an end of the mixing rod 204 arranged in the interior of the cartridge 200.

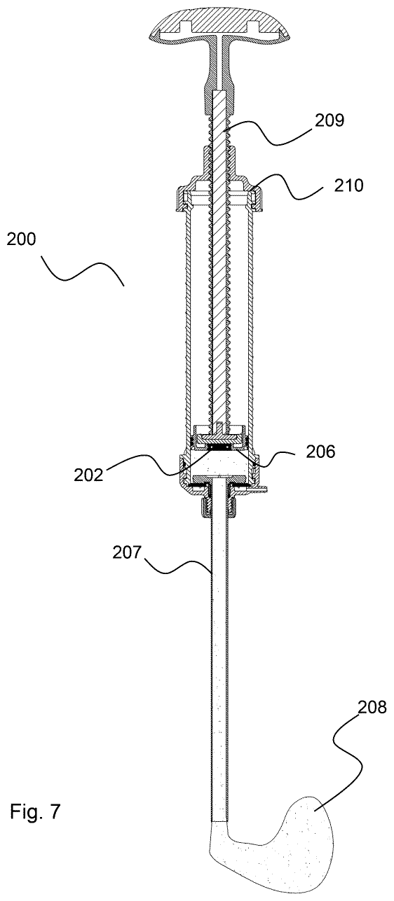

FIG. 7 shows a cartridge 200 comprising a threaded rod 209. The threaded rod 209 engages in a cartridge head 210 so that the threaded rod 209 can be screwed into the cartridge. Thus, a holder 206 for the septum 202 can be moved within the cartridge 200 like a plunger in order to discharge bone cement dough 208 mixed in the cartridge from the cartridge 200 through a discharge tube 207.

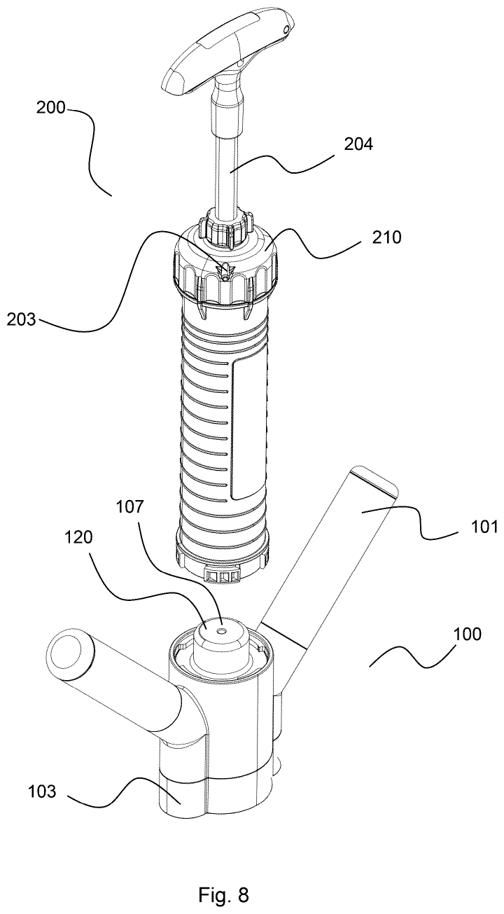

FIG. 8 shows an embodiment of a device according to the invention, which comprises a base part 100 and a cartridge 200 that can be connected to the base part. The base part 100 comprises a screw thread for connection to the cartridge 200. The base part 100 also comprises an outwardly protruding contact region 120 which can be received into the cartridge 200. A plunger 107 and a hollow pin 105 are disposed in this contact region 120. The base part 100 also comprises a bottom part 103 which can stand on a supporting surface during use of the device. The base part 100 also comprises a container 101 for accommodating glass ampules containing monomer liquid. The container 101 comprises a closable opening for receiving a glass ampule. The cartridge 200 comprises a cartridge head 210 with a vacuum port 203. The cartridge 200 also comprises a mixing rod 204 which extends through the cartridge head 210 into the interior of the cartridge 200.

### LIST OF REFERENCE NUMERALS

100 Base part
101 Container
102 Monomer liquid
103 Bottom part
104 Collection region
105 Hollow pin
106 Tip
107 Plunger
108 Feedthrough
109 Spring
110 Holder for hollow pin
111 Conduit element
112 Flexible region of the container
113 Rigid region of the container
114 Vessel
115 Ampule head
116 Stop
117 Filter
118 Carrier
119 Projection
120 Contact region
200 Cartridge
201 PMMA bone cement powder
202 Septum
203 Port for vacuum source
204 Mixing rod
205 Mixing disc
206 Holder for septum
207 Discharge tube
208 Bone cement dough
209 Threaded rod
210 Cartridge head

1. A device for producing bone cement, comprising:
a base part, which comprises a container having a carrier for accommodating a vessel containing monomer liquid, a bottom part having a collection region, a hollow pin having a tip, and a plunger having a feedthrough for accommodating the hollow pin; and
a cartridge for accommodating a PMMA bone cement powder, wherein the cartridge comprises a septum which delimits the cartridge to the outside;
wherein the plunger is configured to accommodate the tip of the hollow pin in the feedthrough when the device is in a basic configuration and to release the tip of the hollow pin from the feedthrough and simultaneously release a monomer liquid from a vessel when the cartridge is pressed onto the plunger.

2. The device according to claim 1, wherein the device is designed and configured to form a fluidic connection between the base part and the cartridge by piercing the septum by means of the hollow pin, in order to transfer a monomer liquid from the collection region into the cartridge.

3. The device according to claim 1, wherein the vessel is a glass ampule or a film bag.

4. The device according to claim 1, wherein the carrier is designed and configured to bring a head of a glass ampule into contact with the plunger.

5. The device according to claim 1, wherein the base part comprises a spring which is arranged and configured to movably support the plunger within the base part.

6. The device according to claim 1, wherein the base part comprises a stop in order to limit the movement of the plunger and/or the cartridge in the base part.

7. The device according to claim 1, wherein the device comprises a filter, wherein the filter is arranged and designed to prevent the ingress of particles from the base part into the cartridge.

8. The device according to claim 1, wherein the cartridge comprises a port for connection to a vacuum source in order to allow a monomer liquid to be transferred from the collection region into the cartridge by means of a pressure difference.

9. The device according to claim 1, wherein the base part comprises a ventilation opening, wherein the ventilation opening is preferably designed as a valve.

10. The device according to claim 1, wherein the septum comprises a rubber-elastic material.

11. The device according to claim 1, wherein the base part is configured to allow a monomer liquid to be transferred from the container into the collection region by free gravitational flow.

12. The device according to claim 1, wherein the container is arranged at an angle to the bottom part which is 20° to 80°, preferably 21° to 55°.

13. The device according to claim 1, wherein the cartridge is detachably connectable to the base part.

14. The device according to claim 1, wherein the base part comprises a conduit element, wherein the conduit element is configured to establish a fluidic connection between the collection region and the hollow pin.

15. The device according to claim 1, wherein the hollow pin is mounted in a cylindrical holder, wherein the holder is arranged in the collection region.

1. The device according to claim 1, wherein the collection region comprises a recess, and wherein the hollow pin is preferably fluidically connected to the lowest point of the recess.

17. The device according to claim 1, wherein the device is designed and configured to transfer a monomer liquid from the base part into the cartridge while the base part stands on a supporting surface such that the bottom part is oriented in the direction of the supporting surface.
